(19) United States
(12) Patent Application Publication
(10) Pub. No.: US 2012/0221059 A1
(43) Pub. Date: Aug. 30, 2012

(54) CERVICAL SPINE CLAMP

(75) Inventors: H. Dennis Mollman, Columbia, MO (US); William Verbarg, Columbia, MO (US); Zachary Hafez, Columbia, MO (US); Nicole Patino, Columbia, MO (US)

(73) Assignee: THE CURATORS OF THE UNIVERSITY OF MISSOURI, Columbia, MO (US)

(21) Appl. No.: 13/162,126

(22) Filed: Jun. 16, 2011

Related U.S. Application Data
(60) Provisional application No. 61/397,980, filed on Jun. 18, 2010.

(51) Int. Cl. A61B 17/70; A61B 17/88

(52) U.S. Cl. 606/277; 606/279

(57) ABSTRACT

A spine immobilization system is provided wherein the system includes a plurality of vertebra clamps structured and operable to be clamped to one of a plurality of target vertebrae, a pair of retaining rails disposable along the length of the target vertebrae between the clamps and each of the target vertebrae, and a pair of clamp interlocking rods structured and operable to interlock the clamps together once each clamp is clamped to the respective one of the target vertebra such that the target vertebrae are securely held together to provide a cumulative, unified vertebral body, whereby the target vertebrae can only move simultaneously together as a single cumulative, unified structure.

### CROSS-REFERENCE TO RELATED APPLICATIONS

This application claims the benefit of U.S. Provisional Application No. 61/397,980, filed on Jun. 18, 2010. The disclosure of the above application is incorporated herein by reference in its entirety.

### FIELD

The present teachings relate to systems for immobilization of the spinal column or vertebral bodies during surgery on the spinal column vertebral bodies.

### BACKGROUND

The statements in this section merely provide background information related to the present disclosure and may not constitute prior art.

Back pain is a significant clinical problem and the surgical and medical costs to treat it are estimated to be over $2 billion per year. It is estimated that seven out of ten people will suffer from cervical spine (neck) pain during their lives. Various conditions can cause such cervical spine pain, such as degeneration of discs, narrowing of the spinal canal, arthritis, and in rare cases, cancer or meningitis. Additionally, trauma to the neck can result in herniation of the discs, whiplash, blood vessel destruction, vertebral injury, and in extreme cases, permanent paralysis. Furthermore, herniated discs or bone spurs may cause a narrowing of the spinal canal or the small openings through which spinal nerve roots exit.

Any of these spinal conditions can cause pressure on the spinal cord in the cervical region, which can be a very serious problem because virtually all of the nerves to the rest of the body have to pass through the cervical region. Hence, trauma or injury to the spinal cord, particularly in the cervical region, can potentially compromise the use and function of generally any organ and/or body part.

A common treatment to relieve the pain and other potential problems caused by such cervical maladies is spinal fusion. Spinal fusion strengthens and stabilizes the spine to alleviate severe and chronic neck pain and to minimize the occurrence of other more serious conditions that can result from trauma to the spinal cord. Generally, spinal fusion is an operation that creates a solid union between two or more vertebrae. For example, cervical spinal fusion creates a solid union between two or more cervical vertebrae, i.e., fuses together two or more cervical vertebrae.

Recent developments in computer assisted spinal surgery, e.g., surgical navigation systems, have improved the accuracy, efficiency and success rate of spinal fusions. However, such surgical procedures on the spine remain difficult and dangerous due to, inter alia, difficulties in keeping the vertebrae of the spine immobilized during such surgeries.

### SUMMARY

Further areas of applicability of the present teachings will become apparent from the description provided herein. It should be understood that the description and specific examples are intended for purposes of illustration only and are not intended to limit the scope of the present teachings.

### DRAWINGS

The drawings described herein are for illustration purposes only and are not intended to limit the scope of the present teachings in any way.

FIG. 1 is an isometric view of a spine immobilization system, in accordance with various embodiments of the present disclosure.

FIG. 2 is a side view of a clamp of the spine immobilization system, shown in FIG. 1, clamped onto a spinal vertebra, in accordance with various embodiments of the present disclosure.

Corresponding reference numerals indicate corresponding parts throughout the several views of drawings.

### DETAILED DESCRIPTION

The following description is merely exemplary in nature and is in no way intended to limit the present teachings, application, or uses. Throughout this specification, like reference numerals will be used to refer to like elements.

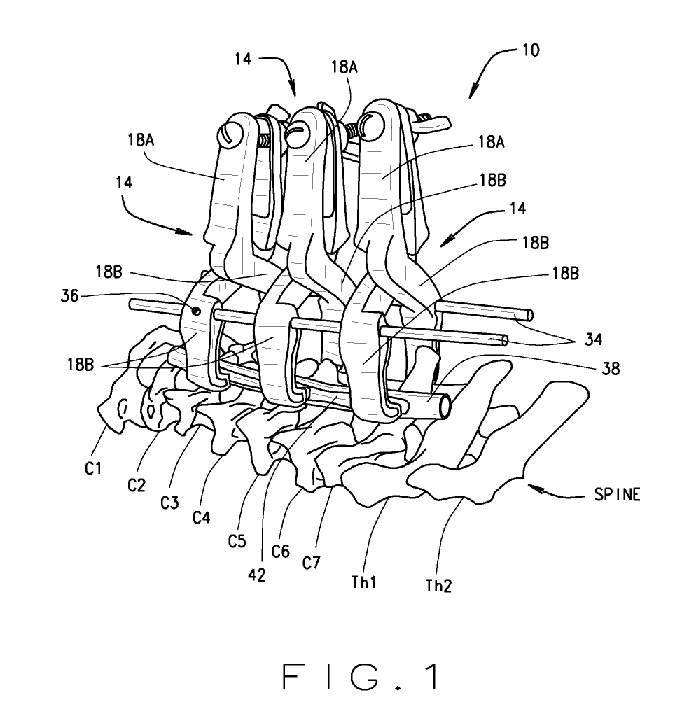

Referring to FIG. 1, in various embodiments, the present disclosure provides a spine immobilization system 10 that is structured and operable to immobilize selected vertebrae during various surgical procedures and reduce the need for multiple X-ray scans, e.g., 360° or 18020 three-dimensional scans, during the procedure. Particularly, the spine immobilization system 10 is structured and operable to improve the accuracy and efficiency of the surgical procedure, reduce the trauma to the patient, and reduce the patient's exposure to radiation.

Generally, the spine immobilization system 10 is connectable to two or more vertebra such that the vertebrae are held in a desired constant position and orientation, i.e., a desired alignment, with respect to each other. More specifically, the spine immobilization system 10 clamps and steadfastly holds selected vertebra together such that each respective vertebra can no longer move independently, but rather the selected vertebrae are securely held together to provide a cumulative, unified vertebral body, whereby the selected vertebrae can only move simultaneously together as a single cumulative, unified structure. Therefore, the respective vertebrae of the unified vertebral body can undergo a surgical procedure without the risk or consequences of any of the vertebra moving independently during the procedure.

Although FIG. 1 exemplarily illustrates the spine immobilization system 10 being utilized to immobilize several cervical vertebrae, particularly cervical vertebrae C3, C4, C5, C6 and C7, it should be understood that the spine immobilization system 10 can be implemented to immobilize any a group of thoracic vertebrae, or a group of lumbar vertebrae, or any group of adjacent vertebra from adjoining sections of the spine.

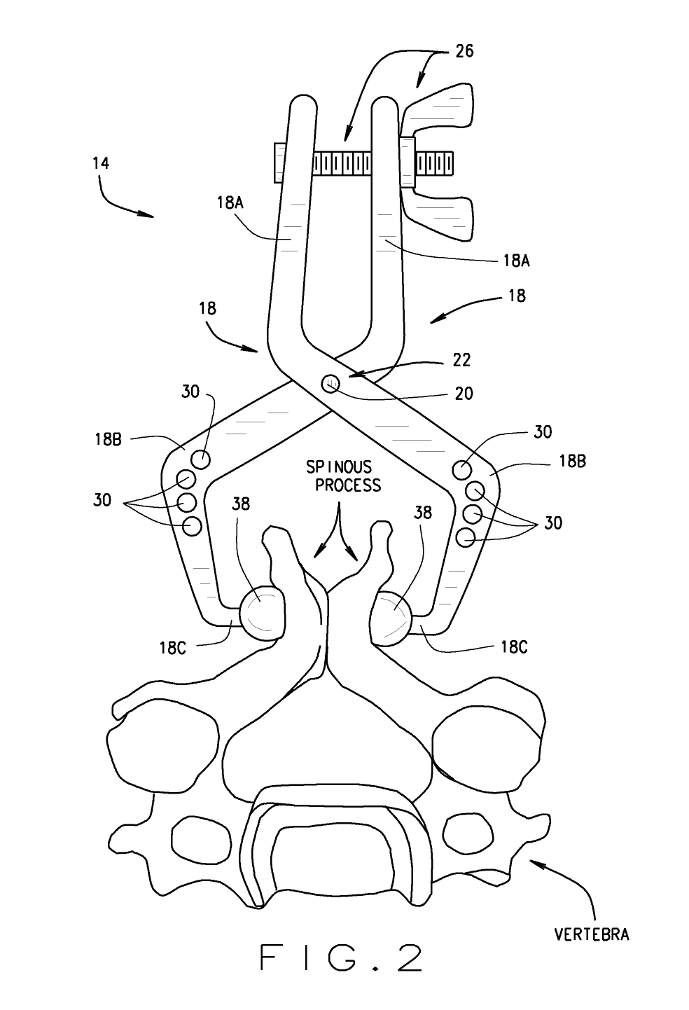

Referring now to FIGS. 1 and 2, the spine immobilization system 10 includes a plurality of vertebra clamps 14. Particularly, the spine immobilization system 10 can include two, three, four or more clamps 14 depending on the number of vertebrae to be immobilized, as described below. Each clamp 14 includes a pair of opposing arms 18 that are pivotally connected at a center point 22 in a scissor-like fashion via a connecting device 20. The connecting device 20 of each clamp 14 can be any device suitable to securely pivotally connect the arms 18 of each clamp 14 to each other while providing a fulcrum about which each respective arm 18 can pivot, e.g., a screw, a rivet, a pin, a nut and bolt, etc.

Each arm 18 comprises a handle 18A and a jaw 18B extending from the respective handle 18A. Each jaw 18B includes a foot 18C formed at the distal end. Additionally, each arm 18 is somewhat S shaped such that the respective clamp 14 opens and closes in a scissor-like fashion. That is, when the opposing handles 18A of a particular clamp 14 are spread, i.e., moved away from each other, the arms 18 pivot about the connecting device 20 causing the jaws 18B to open, i.e., move away from each other. Conversely, when the opposing handles 18A of a particular clamp 14 are squeezed, i.e., moved toward each other, the arms 18 pivot about the connecting device 20, which operates as a fulcrum, causing the jaws 18B to close, i.e., move toward each other. Accordingly, a portion of a selected vertebra, e.g., the spinous process of a selected vertebra, can be grasped between the jaws 18B of a respective clamp 14 to install the spine immobilization system 10, as described below.

Additionally, each clamp 14 includes an adjustable tightening mechanism 26 that is structured and operable to maintain the handles 18A of the respective clamp 14 in a desired position with respect to the distance between the handles 18A of the respective clamp 14. More specifically, the tightening mechanism 26 of each clamp 14 is structured and operable to tighten the jaws 18B about a portion of a selected vertebra and maintain a desired grip, i.e., pressure or force applied between the opposing jaws 18B to the selected vertebra, such that the respective clamp 14 is steadfastly clamped to the selected vertebra. Thus, the jaws 18B of a clamp 14 can be opened, and placed about the desired portion of the selected vertebra, e.g., the spinous process, and closed to grasp the portion of the vertebra between the feet 18C of the jaws 18B. Then, while holding the clamp 14 in a desired position, the tightening mechanism 26 can be operated to squeeze the handles 18A further together, thereby causing the jaws 18B to tighten their grip on the vertebra. Moreover, the tightening mechanism 26 is operable to provide a desired amount of force between the jaw feet 18C, e.g., 60 ft/lbs to 80 ft/lbs. Hence, each clamp 14 can be clamped onto the vertebra and adjusted, via the tightening mechanism 26, to apply a particular amount of force on the portion of the vertebra clamped/grasped between the clamp jaws 18B.

The tightening mechanisms 26 can be any mechanism structured and operable to hold the handles 18A of the respective clamp 14 in a desired position relative to each other such that the desired amount of force is exerted and maintained on the portion of the vertebra clamped/grasped between the jaws 18B. For example, in various embodiments, the tightening mechanism 26 can be a threaded bolt and nut, whereby the bolt extends through distal ends of the handles 18A and the nut can be turned, i.e., tightened, by hand or with the assistance of a tool, e.g., a wrench, to squeeze the handles 18A together until the desired amount of force is generated between the jaws 18B. As exemplarily illustrated in FIG. 2, in various implementations of such embodiments, the nut can be a wing nut that can be tightened or loosed by hand, without the use of a tool.

Alternatively, the tightening mechanism 26 can be a screw that extends through one handle 18A and is threaded into the opposing handle 18A, whereby the screw can be turned to squeeze the handles 18A together until the desired amount of force is generated between the jaws 18B. In yet other embodiments, it is envisioned that the tightening mechanism 26 can be a tension locking device similar to that used in vice grips, or a ratchet locking system similar to that used in contemporary manual caulking guns, or any other suitable manually operated or automated mechanism. Additionally, in various embodiments, it is envisioned that each clamp 14 can include a mechanical or electronic force gauge (not shown) that is operable to indicate the amount of force that is being applied to the vertebra between the jaws 18B.

The spine immobilization system 10 further includes a pair of clamp interlocking rods 34, and each jaw 18B of each clamp 14 includes one or more locking apertures 30 that are sized to receive the interlocking rods 34. The interlocking rods 34 are rigid and inflexible such that they cannot be bent without applying substantial force. As described further below, once the clamps 14 have been clamped onto the respective vertebrae with the desired amount of force applied between the jaws 18B, one interlocking rod 34 is inserted through a selected locking aperture 30 in the jaw 18B on one side of each of the clamps 14. Subsequently, the other interlocking rod 34 is inserted through a selected locking aperture 30 in the each of the opposing jaws 18B.

As one skilled in the art will readily recognize, spinal anatomy is such that any group of adjacent vertebrae are not necessarily aligned in a straight line, nor is the shape of adjacent vertebrae necessarily identical. Therefore, the clamps 14 will not necessarily be clamped onto each vertebra at exactly the same location and/or orientation. Each clamp 14 may need to be moved up or down, left or right, or rotated differently with respect to the clamps 14 when clamped onto the respective vertebra. Hence, as a result of varying location and orientation of each clamp 14 when clamped onto the respective vertebrae, and natural curvature of the spine, after all the individual clamps 14 are installed, they will not necessarily be linearly aligned.

To compensate for the varying location and orientation of each clamp 14, as described above, in various embodiments, each clamp 14 can include a plurality of locking apertures 30 formed in predetermined pattern within each jaw 18B. Therefore, once the clamps 14 are installed, the rigid inflexible interlocking rods 34 can be inserted through suitably aligned locking apertures 30 in each clamp 14 to interlock the clamps 14 together. Alternatively, in various embodiments, the interlocking rods 34 can be formed to have a predetermined curvature similar to the curvature of the spine at the point that the spine immobilization system 10 is to be utilized. Therefore, once the clamps 14 are installed, the rigid inflexible interlocking rods 34, having the desired curvature, can be inserted through locking apertures 30 in each clamp 14 to interlock the clamps 14 together.

Furthermore, in various embodiments, the locking apertures 30 are sized to have a friction fit with the interlocking rods 34 such that when the interlocking rods 34 are inserted through the locking apertures 30 the clamps 14 will not readily move along the length of the interlocking rods 34. Therefore, all the clamps 14 will be interlocked together providing a single cumulative and unified structure, i.e., the spine immobilization system 10. In various other implementations, each clamp 14 can include rod securing devices 36 (exemplarily illustrated on one of the clamps 14 in FIG. 1) that fixedly secure the interlocking rods 34 within the locking apertures 30 such that when the interlocking rods 34 are inserted through the locking apertures 30 the clamps 14 will not readily move along the length of the interlocking rods 34. For example, it is envisioned that such securing devices could comprise a plurality of set screws disposed with the jaws 18B of each clamp such that each set screws extend from the exterior of each jaw 18B into the interior of a respective one of the locking apertures 30. Once the interlocking rods 34 are inserted through the locking apertures 30 the appropriate set screws can be tightened to lock down on the respective interlocking rod 34 such that the clamps 14 will not move along the length of the interlocking rods 34. Therefore, all the clamps 14 will be interlocked together providing a single cumulative and unified structure, i.e., the spine immobilization system 10.

The spine immobilization system 10 further includes a pair of conformable retaining rails 38. As described further below, the retaining rails 38 are disposable between the jaw feet 18C of each clamp 14 and the respective vertebra to which the clamps 14 are attached. Additionally, the retaining rails 38 are structured and operable to conform to the shape of the vertebra in order to provide a ‘form fitting grip’ on the clamped vertebrae to cradle the vertebrae and ensure that no damage is done to the vertebrae and surrounding anatomy. Furthermore, the retaining rods 38 are structured and operable to securely and steadfastly retain any interstitial vertebra, i.e., vertebrae located between the vertebrae that are directly clamped by a clamp 14, in alignment with the directly clamped vertebrae to form the cumulative, unified vertebral body.

Particularly, the retaining rails 38 are fabricated from a bio-compatible material and are structured to be stiff but slightly flexible and/or bendable along its length, while being compressive and conformable along its exterior surface. Hence, when disposed between the jaw feet 18C of each clamp 14 and the respective vertebra to which the clamps 14 are attached, the retaining rails 38 will substantially conform to the shape of the portion of each vertebra to which a clamp 14 is attached. This provides a secure grip on each directly clamped vertebra such that each respective vertebra is steadfastly retained by the spine immobilization system 10. Particularly, the flexibility and conformability of the retaining rails 38 allow the retaining rails 38 to conform to the shape of each respective vertebra and to the natural curvature of the spine, and the rigidity of the retaining rails 38 provides stability among the clamped vertebrae. Additionally, when the spine immobilization system 10 is installed, the retaining rails 38 will contact and substantially conform to the shape of any interstitial vertebra. Thus, the retaining rails 38 will securely and steadfastly retain the interstitial vertebrae in alignment with the directly clamped vertebrae and will further provide stability and immobility among all the vertebrae to which the spine immobilization system 10 is attached.

In various embodiments, the retaining rails 38 can include a positioning channel 42 that is structure and operable to receive the jaw feet 18C of the clamps 14. Particularly, the positioning channels 42 are structured and operable to properly position the jaw feet 18C in contact with the retaining rails 38 and assist in retaining the retaining rails 38 in a desired orientation between the jaw feet 18C and the respective vertebra. That is, the positioning channels 42 maintain the retaining rails squarely centered between the jaw feet 18C and the respective vertebra.

Implementation, i.e., installation, of the spine immobilization system 10 will now be described. Initially, the spine is dissected and the portion of the spine comprising the target vertebrae, i.e., the vertebrae to be clamped together using the spine immobilization system 10, is exposed. Then, in various implementations, the retaining rails 38 are disposed along the portion of target vertebrae to which the clamps 14 are to be attached. For example, the retaining rails 38 can be disposed along the side of the spinous process, or along the lamina, of the target vertebrae. Although it will be understood that the clamps 14 can be clamped any suitable portion of target vertebrae, for simplicity and clarity, the clamps 14 will be described herein as being clamped to the sides of spinous process, or the lamina, of each target vertebra, as exemplarily illustrated in FIGS. 1 and 2.

Once the retaining rails 38 have been placed along the sides of the spinous processes, or the lamina, of the target vertebrae, a first one of the clamps 14 is placed over the spinous process of a first one of the target vertebrae, e.g., one of the vertebra at the end of the row of the target vertebrae, such that each of the feet 18C of the opposing jaws 18B is adjacent a respective one of the retaining rails 38. For exemplary purposes, the first one of the target vertebrae will be referred to herein as vertebra C7. The tightening mechanism 26 of the clamp 14 is then operated to close the jaws 18B bringing the feet 18C into contact with the retaining rails 38. The jaws 18B are then tightened, via the tightening mechanism 26, compressing the retaining rails 38 between the feet 18C and the sides of the spinous process of the vertebra until the desired amount of force is applied to spinous process between the jaws, e.g., 60 ft/lbs to 80 ft/lbs. As described above, the retaining rails 38 will conform to the shape of the sides of the spinous process such that the respective vertebra is securely clasped between jaws 18B of the clamp 14. In various embodiments, the retaining rails 38 can include a longitudinal groove structured to receive the feet 18C of the clamp jaws 18B, thereby assisting maintaining the clamp feet 18C in a desired contact position with the retaining rails 38 as the clamp 14 is tightened.

After the first clamp 14 is installed on the first one of the target vertebrae, e.g., vertebra C7, a second one of the clamps 14 is clamped to the spinous process of a second one of the target vertebrae as described above with regard to the first one of the target vertebrae. The second one of the target vertebrae can the can be a vertebra immediately adjacent the first one of the target vertebrae or it can be separated from the first one of the target vertebrae by one or more interstitial vertebra. For example, if the first one of the target vertebrae is C7, the second one of the target vertebrae can be C6 or it can be C5 or it can be C4, etc. As described above, the spine immobilization system 10 can include two or more clamps 14, depending on the number of target vertebrae, i.e., the number of vertebrae that are to be immobilized. Hence, if a third or fourth one of the clamps 14 are to be utilized, they would be clamped to the spinous process of a third or fourth one of the target vertebrae, in the same manner as described above with regard to the first one of the target vertebrae.

Although the installation method has been described above wherein the retaining rails 38 are placed along the sides of the spinous processes of the target vertebrae prior to clamping the clamps 14 onto the spinous processes, it is envisioned that in various embodiments, the retaining rails can be connected to the jaw feet 18C of the clamps 14 prior to clamping the clamps 14 onto the spinous processes. Therefore, as each clamp 14 is positioned about the respective spinous process, the retaining rails 38 will simultaneously be positioned on the sides of the spinous process and compressed between the clamps jaws 18B of each clamp 14 and spinous processes of the respective vertebra as each clamp 14 is tightened, via the respective tightening mechanism 26.

Once each clamp 14 has been clamped onto the respective vertebra, a first one of the clamp interlocking rods 34 is inserted through a selected one of the locking apertures 30 in a first one of the jaws 18B of each of the clamps 14, in the embodiments wherein there are a plurality of locking apertures 30 in each clamp jaw 18B. Or, in the embodiments wherein each jaw 18B of each clamp 14 comprises a single locking aperture 30, a first one of the clamp interlocking rods 34 is inserted through the locking aperture 30 in a first one of the jaws 18B of each of the clamps 14. Subsequently, the second one of the clamp interlocking rods 34 is inserted through a respective one of the locking aperture(s) 30 in the second one of the jaws 18B of each of the clamps 14.

Inserting the clamp interlocking rods 34 through the locking aperture 30 interlocks all of the clamps 14 together such that clamps 14, retaining rails 38 and interlocking rods 34 become a unified immobilizing structure that clamps and steadfastly holds the target vertebra together such that each respective vertebra can no longer move independently. Rather, the target vertebrae are securely held together to provide a cumulative, unified vertebral body, whereby the target vertebrae can only move simultaneously together as a single cumulative, unified structure. Therefore, target vertebrae are joined in a constant cumulative formation and can undergo imaging and a surgical procedure without the risk or consequences of any of the target vertebrae moving independently during the procedure.

Importantly, all components of the spine immobilization system 10 described herein are fabricated of X-ray translucent materials or fluoroscopic imaging-compatible material. Particularly, all components of the spine immobilization system 10 described herein are fabricated of any appropriate material or combination of materials, such as plastics, nylons, composites, metals, polymers, rubbers and the like that will not interfere with medical imaging systems, i.e., that will not cause starburst artifacts in X-rays or other types of fluoroscopic images.

It is envisioned that, in various embodiments, after the retaining rails 38 have been disposed along the sides of the spinous processes of the target vertebrae (or any other desired portion of each target vertebrae) the clamps 14 can be disposed along the retaining rails 38 between the target vertebrae such that no direct force is applied to any of the target vertebrae by the clamps 14, Rather, force from the tightened clamps 14 is transferred along the retaining rails 38 and the target vertebrae are immobilized and held together as a cumulative, unified vertebral body by squeezing the target vertebrae between the retaining rails 38.

Additionally, it is envisioned that the spine immobilization system 10, described herein, can be utilized for any surgical procedure where the spine must be stabilized, e.g., multiple vertebral fusion or any other procedure. Additionally, it is envisioned that spine immobilization system 10, described herein, can be utilized on any creature or organism having a spine, including any human or non-human animals. Additionally, it is envisioned that the clamps 14 can vary in size, i.e., be larger or smaller, wider or narrower, thicker or thinner, taller or shorter, to accommodate any size vertebrae.

While the present disclosure has described various specific embodiments, it will be understood that the described systems and methods are capable of further modifications. This patent application is intended to cover any variations, uses, or adaptations of the present disclosure following, in general, the principles of the present disclosure and including such departures as come within known or customary practice within the art to which the present disclosure pertains and as may be applied to the essential features herein set forth above and as follows in scope of the appended claims.

What is claimed is:

1. A spine immobilization system for immobilizing a plurality of adjacent vertebrae during imaging and surgery, said system comprising:
a plurality of vertebra clamps, each clamp structured and operable to be clamped to a portion of a corresponding one of a plurality of target vertebrae, the target vertebrae being a group of adjacent vertebrae selected to undergo an imaging and/or surgical procedure;
a pair of retaining rails disposable along the length of the target vertebrae between the clamps and each of the corresponding ones of the target vertebrae, the retaining rods structured and operable to conform to the shape of the target vertebrae and maintain stability and alignment of the target vertebra once the clamps are clamped to the corresponding ones of the target vertebrae; and
a pair of clamp interlocking rods structured and operable to interlock the clamps together such that the target vertebrae are securely held together to provide a cumulative, unified vertebral body, whereby the target vertebrae can only move simultaneously together as a single cumulative, unified structure.

2. The system of claim 1, wherein each clamp comprises:
a pair of pivotally connected opposing jaws that are structured and operable to open such that each respective clamp can be positioned about the portion of the corresponding one of a plurality of target vertebrae to be clamped, and to close such the jaws will grasp the corresponding one of a plurality of target vertebrae; and
a tightening mechanism that is structured and operable to clamp each clamp to the portion of a corresponding one of a plurality of target vertebrae with a selected specific amount of force applied between the opposing jaws to the portion of the corresponding one of a plurality of target vertebrae.

3. The system of claim 2, wherein each of the opposing jaws of each clamp comprises at least one locking aperture structured and operable to have one of the interlocking rods inserted therethrough such that each of the clamps are interlocked with the other clamps.

4. The system of claim 3, wherein each locking aperture is sized to have a friction fit with the interlocking rods such that when the interlocking rods are inserted through the locking apertures the clamps will not readily move along the length of the interlocking rods.

5. The system of claim 3, wherein each of the opposing jaws of each clamp further comprises a rod securing device adjacent each locking aperture that is structured and operable to fixedly secure the interlocking rods within the locking apertures such that when the interlocking rods are inserted through the locking apertures the clamps will not readily move along the length of the interlocking rods.

6. The system of claim 1, wherein each of the retaining rails is fabricated from conformable material that is operable to conform to the shape of the portion of each vertebra to which the respective clamp is clamped.

7. The system of claim 1, wherein each retaining rails includes a positioning channel structured and operable to receive a foot of each clamp jaw to properly position the jaw foot in contact with the respective retaining rail and assist in retaining the respective retaining rail between the jaw foot and the respective vertebra.

8. The system of claim 1, wherein the clamps, the retaining rails and the interlocking rods are fabricated from a bio-compatible material.

9. The system of claim 1, wherein the clamps, the retaining rails and the interlocking rods are fabricated from a fluoroscopic imaging-compatible material.

10. A spine immobilization system for immobilizing a plurality of adjacent vertebrae during imaging and surgery, said system comprising:
a plurality of vertebra clamps, each clamp structured and operable to be clamped to a portion of a corresponding one of a plurality of target vertebrae, the target vertebrae being a group of adjacent vertebrae selected to undergo an imaging and/or surgical procedure, each clamp comprising:
a pair of pivotally connected opposing jaws that are structured and operable to open such that each respective clamp can be positioned about the portion of the corresponding one of the target vertebrae to be clamped, and to close such the jaws will clamp onto the corresponding one of a plurality of target vertebrae; and
a tightening mechanism that is structured and operable to clamp each clamp to the portion of a corresponding one of a plurality of target vertebrae with a selected specific amount of force applied between the opposing jaws to the portion of the corresponding one of a plurality of target vertebrae;
a pair of retaining rails disposable along the length of the target vertebrae between the clamp jaws and each of the corresponding ones of the target vertebrae, the retaining rods structured and operable to conform to the shape of the target vertebrae and maintain stability and alignment of the target vertebra once the clamps are clamped to the corresponding ones of the target vertebrae; and
a pair of clamp interlocking rods structured and operable to interlock the clamps together such that the target vertebrae are securely held together to provide a cumulative, unified vertebral body, whereby the target vertebrae can only move simultaneously together as a single cumulative, unified structure.

11. The system of claim 10, wherein each of the opposing jaws of each clamp comprises at least one locking aperture structured and operable to have one of the interlocking rods inserted therethrough such that each of the clamps are interlocked with the other clamps.

12. The system of claim 11, wherein each locking aperture is sized to have a friction fit with the interlocking rods such that when the interlocking rods are inserted through the locking apertures the clamps will not readily move along the length of the interlocking rods.

13. The system of claim 11, wherein each of the opposing jaws of each clamp further comprises a rod securing device adjacent each locking aperture that is structured and operable to fixedly secure the interlocking rods within the locking apertures such that when the interlocking rods are inserted through the locking apertures the clamps will not readily move along the length of the interlocking rods.

14. The system of claim 10, wherein each of the retaining rails is fabricated from conformable material that is operable to conform to the shape of the portion of each vertebra to which the respective clamp is clamped.

15. The system of claim 10, wherein each retaining rails includes a positioning channel structured and operable to receive a foot of each clamp jaw to properly position the jaw foot in contact with the respective retaining rail and assist in retaining the respective retaining rail between the jaw foot and the respective vertebra.

16. The system of claim 10, wherein the clamps, the retaining rails and the interlocking rods are fabricated from at least one or a bio-compatible material and a fluoroscopic imaging-compatible material.

17. A method for immobilizing a plurality of adjacent vertebrae during imaging and surgery, said method comprising:
disposing a pair of retaining rails of an spine immobilization system along opposing sides of a plurality of target vertebrae, the target vertebrae being a group of adjacent vertebrae selected to undergo an imaging and/or surgical procedure,
placing each of a plurality of clamps of the spine immobilization system over a corresponding one of the target vertebrae, each clamp comprising:
a pair of pivotally connected opposing jaws, each jaw including a locking aperture and having a foot disposed at a distal end, the opposing jaws structured and operable to open such that each clamp can be positioned over a corresponding one of the target vertebrae such that each respective jaw foot can be positioned adjacent one of the retaining rails disposed along opposing sides of the target vertebrae, and to close such the jaw feet will clamp onto the corresponding one of a plurality of target vertebrae; and
a tightening mechanism that is structured and operable to close the respective clamp to clamp each respective clamp to the portion of a corresponding one of a plurality of target vertebrae with a selected specific amount of force applied between the opposing jaw feet to the corresponding one of a plurality of target vertebrae;
operating the tightening mechanism of each clamp to close the respective jaws such that each foot of each jaw of each respective clamp is brought into contact with a corresponding one of the retaining rails, thereby compressing the retaining rails between the jaw feet and the corresponding one of a plurality of target vertebrae, and operating the tightening mechanism such that the selected specific amount of force is applied between the jaw feet to the corresponding one of a plurality of target vertebrae; and
inserting a first one of a pair of clamp interlocking rods of the of the spine immobilization system through the locking aperture in corresponding jaws of each of the clamps, and inserting a second one of the pair of clamp interlocking rods through the locking aperture in the corresponding opposing jaws of each of the clamps, thereby interlocking all of the clamps together such that the clamps, retaining rails and interlocking rods become a unified immobilizing structure that clamps and steadfastly holds the target vertebra together such that each respective vertebra can no longer move independently.

18. The method of claim 17, wherein disposing the retaining rails along opposing sides the target vertebrae comprising disposing along opposing sides of the target vertebrae retaining rails fabricated from conformable material that is operable to conform to the shape of the portion of each vertebra to which the respective clamp is clamped.

19. The method of claim 17, wherein disposing the retaining rails along opposing sides the target vertebrae comprising disposing along opposing sides of the target vertebrae retaining rails comprising a positioning channel structured and operable to receive the feet of the clamp jaws to properly position the jaw feet in contact with the respective retaining rail and assist in retaining the respective retaining rail between the jaw feet and the respective vertebra.

20. The method of claim 17, wherein the clamps, the retaining rails and the interlocking rods are fabricated from at least one or a bio-compatible material and a fluoroscopic imaging-compatible material.
